(19) United States
(12) Patent Application Publication
(10) Pub. No.: US 2026/0157625 A1
(43) Pub. Date: Jun. 11, 2026

(54) SYSTEM AND METHOD FOR SELECTING ABERROMETER FRAMES FOR MEASURING REFRACTIVE ERROR

(71) Applicant: Alcon Inc., Fribourg (CH)

(72) Inventors: Hector Mas Peris, Mission Viejo, CA (US); Zhaokai Meng, Aliso Viejo, CA (US)

(21) Appl. No.: 19/373,193

(22) Filed: Oct. 29, 2025

Related U.S. Application Data
(60) Provisional application No. 63/729,899, filed on Dec. 9, 2024.

(51) Int. Cl. A61B 3/103; A61B 3/00; A61B 3/107; A61B 3/113; A61B 3/14; G16H 50/30

(52) U.S. Cl. CPC A61B 3/103; A61B 3/0025; A61B 3/107; A61B 3/113; A61B 3/14; G16H 50/30

(57) ABSTRACT

A system includes an aberrometer configured to measure an eye of a patient, a computing device, and possibly one or more other imaging modalities. The computing device is configured to receive frames from the aberrometer and calculate noise data from at least one of (a) the frames and (b) images of the eye obtained using an imaging modality. The computing device processes the noise data, such as using a machine learning model, to obtain labels for the frames and selects a selected frame from the frames according to the labels. The computing device processes the selected frame to obtain a refractive error of the eye.

### TECHNICAL FIELD

The present disclosure relates generally to methods for selecting aberrometer frames for measuring refractive error.

### BACKGROUND

Light received by the human eye passes through the transparent cornea covering the iris and pupil of the eye. The light is transmitted through the pupil and is focused by a crystalline lens positioned behind the pupil in a structure called the capsular bag. The light is focused by the lens onto the retina, which includes rods and cones capable of generating nerve impulses in response to the light.

Refractive error is caused by the inability of the cornea and lens to properly focus light on the retina. One highly accurate way to measure refractive error is with an aberrometer. Some aberrometers project light into the eye in the form of, or in order to simulate, a planar wavefront entering the eye and measure the phase of light reflected out of the eye in order to characterize the refractive error of the eye. To obtain a characterization of refractive error, measurements of received light typically undergo extensive processing.

### BRIEF SUMMARY

The present disclosure relates generally to ophthalmic methods and systems for selecting which frame of measurements from an aberrometer to use for calculating refractive error.

Particular embodiments disclosed herein provide a system including an aberrometer configured to measure an eye of a patient and a computing device. The computing device is configured to: receive frames from the aberrometer; calculate noise data from at least one of (a) the frames and (b) images of the eye obtained using an imaging modality; process the noise data using a machine learning model to obtain labels for the frames; select a selected frame from the frames according to the labels; process the selected frame to obtain a refractive error of the eye; and output the refractive error.

The following description and the related drawings set forth in detail certain illustrative features of one or more embodiments.

### BRIEF DESCRIPTION OF THE DRAWINGS

The appended figures depict certain aspects of the one or more embodiments and are therefore not to be considered limiting of the scope of this disclosure.

FIG. 1 illustrates anatomy of a human eye.

FIG. 2 illustrates imaging of an eye using an ophthalmic imaging device including an aberrometer, in accordance with certain embodiments.

FIG. 3 illustrates an approach for training a machine learning model to characterize outputs of an aberrometer, in accordance with certain embodiments.

FIG. 4 is a process flow diagram of a method for selecting frames from the output of an aberrometer, in accordance with certain embodiments.

FIG. 5 illustrates an example computing device that implements, at least partly, one or more functionalities for selecting frames from the output of an aberrometer, in accordance with certain embodiments.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

As described above, aberrometers project light into the eye in the form of, or in order to simulate, a planar wavefront entering the eye and measure the phase of light reflected out of the eye in order to characterize the refractive error of the eye. To obtain a characterization of refractive error, measurements of received light typically undergo extensive processing, which requires extensive hardware and processing power (e.g., compute cycles, memory, etc.). An aberrometer typically outputs frames, where each “frame” refers to one or more measurements of phase and/or intensity of light detected by the aberrometer. The frame is further processed to derive a wavefront refractive error, which may characterize the degree of spherical error (e.g., myopia or hyperopia), cylindrical error (e.g., astigmatism), and high order aberration error of the measured eye. The processing required is quite complex and may take much longer than the measuring period (e.g., inverse of the frame rate) of the aberrometer.

Accordingly, particular embodiments of the present disclosure provide an approach for selecting a subset of frames for processing to obtain a corresponding wavefront refractive error. In particular, due to movement of an eye during measurement, the refractive error calculated from aberrometer measurements may be inaccurate. Typically, the frame rate at which frames are captured is much higher than the rate at which refractive error may be calculated from the frames. Accordingly, using the approach described herein a subset of frames may be selected for further processing. In some embodiments, a machine learning model is trained to process noise relating to movement of the eye to estimate noise in refractive error calculations based on corresponding frames from the aberrometers. Using the estimated noise, frames may be selected for subsequent processing. Therefore, the embodiments described herein provide a technical solution to the technical problem described above, by processing only a subset of frames output by an aberrometer to obtain a characterization of refractive error, which allows for using less processing resources (e.g., compute cycles).

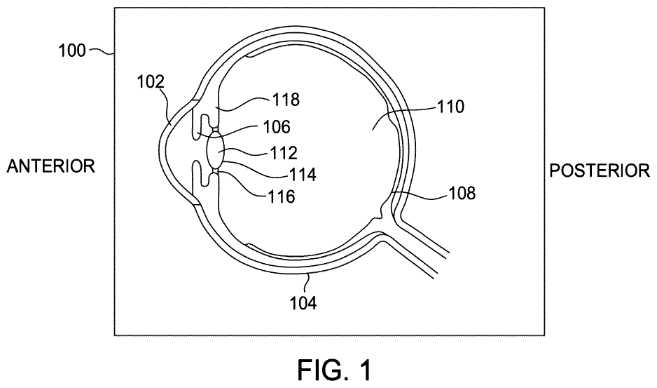

FIG. 1 is a diagram illustrating parts of the human eye 100 that may be understood with respect to the anterior side, through which light enters the eye, and the posterior side opposite the anterior side. At the anterior side of the eye 100, a thin transparent layer known as the cornea 102 is linked to the sclera 104, which forms the generally spherical wall of the eye 100. The cornea 102 and sclera 104 are connected by a ring called the limbus. The iris 106, the color of the eye, and an opening defined by it, the pupil, are positioned behind the cornea and are visible due to the cornea's 102 transparency. The retina 108 is formed on an interior surface of the sclera 104 opposite the cornea 102 and iris 16. The volume defined by the sclera 104 is occupied by the transparent jelly of the vitreous body 110.

The crystalline lens 112 is a transparent, biconvex structure in the eye that, along with the cornea 102, helps to refract light to be focused on the retina 108. The lens 112, by changing its shape, functions to change the focal distance of the eye so that it can focus on objects at various distances, thus allowing a sharp real image of the object of interest to be formed on the retina 108. This adjustment of the lens 112 is known as accommodation, and is similar to the focusing of a photographic camera via movement of its lenses.

The lens 112 is positioned behind the iris 106 in a capsular bag 114. The capsular bag 114 is attached at its perimeter to the suspensory ciliary ligament 116. The ciliary ligament 116 attaches the capsular bag 114 to the ciliary body 118. The ciliary body 118 is a ring-shaped muscle that attaches the ciliary ligament 116 to the sclera 104 and which can contract or relax in order to change the shape of the lens 112.

The lens 112 and cornea 102 cooperate to focus light on the retina in an emmetropic eye 100. The focus of the light may also be in front of the retina in a myopic eye, beyond the retina in an hyperopic eye, or may be spread along an axis (astigmatism). Deviation of the focus from a single diffraction-limited point on the retina is known as refractive error and may be measured subjectively by an optometrist or objectively using a device such as refractometer or aberrometer.

Objective measurements of refractive error may be affected by movement of the eye 100 during measurement. Accordingly, in the approach described below, the amount of movement of the eye 100 may be characterized and related to error in objective measurements of refractive error. The examples below are described with reference to an aberrometer with the understanding that measurements obtained using any instrument for measuring refractive error may also be used.

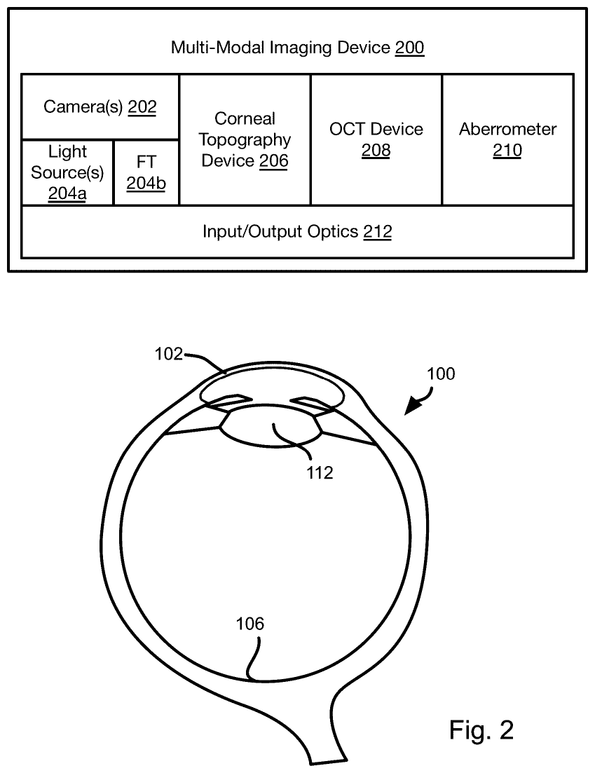

Referring to FIG. 2, imaging of the eye 100 may be performed using a single- or multi-modal imaging device. For example, the illustrated multi-modal imaging device 200 may be used or one or more single-model imaging devices each performing any one of the imaging modalities (e.g., CT device 206, OCT device 208, aberrometer 210, camera 202, etc.) of the multi-modal imaging device 200 may be used.

The multi-modal imaging device 200 may include one or more cameras 202, such as a visible light camera. One or more light sources 204a may illuminate the eye 100 to facilitate capturing images with the one or more cameras 202. A fixation target (FT) 204b may be incorporated and displayed to the eye 100 in order to direct the gaze of the eye 100 to a desired orientation relative to an optical axis of the one or more cameras 202. The one or more cameras 202 may implement a three-dimensional camera system that is configured to capture three-dimensional position coordinates of an object. For example, the one or more cameras 202 and one or more light sources 204a may be implemented as the NGENUITY 3D VISUALIZATION SYSTEM provided by Alcon Inc. of Fort Worth Texas.

The multi-modal imaging device 200 may include a corneal topography device 206. The corneal topography device 206 measures the shape of the cornea 102 in order to estimate the diffractive power of the cornea 102 and any refractive error of the cornea 102, e.g., astigmatism. The corneal topography device 206 may measure the contours of the inner and outer surfaces of the cornea 102 in order to perform the function thereof.

The multi-modal imaging device 200 may include an optical coherence tomography (OCT) device 208. The OCT device 208 obtains a volumetric image of the eye 100, including one or both of the anterior chamber (region between the cornea 102 and the capsular bag 114) and the retina 108.

The multi-modal imaging device 200 may include an aberrometer 210, such as a wavefront aberrometer, that is configured to measure refractive error of the eye 100, including the combined refractive properties of the cornea 102, lens 112 (or intra-ocular lens (IOL)), and the axial length of the eye 100.

The various imaging devices of the multi-modal imaging device 200 may use input/output optics 212 to transmit light to the eye 100 and receive light reflected from the eye. The input/output optics 212 may include one or more lenses and/or beam splitters for routing light to the various imaging devices. Alternatively, each imaging device may have its own input/output optics.

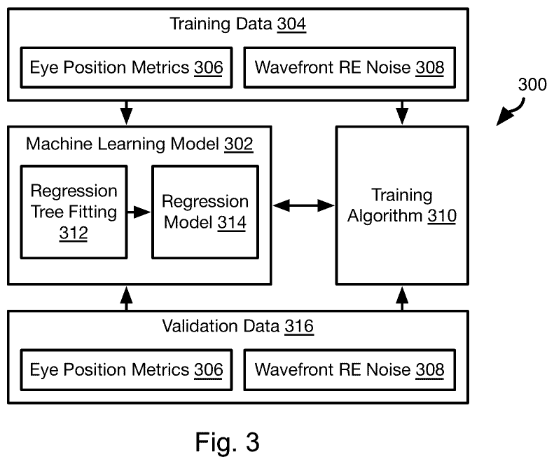

Referring to FIG. 3, the illustrated system 300 may be used to characterize noise that may be used to select frames output by an aberrometer 210 for further processing. As described above, in existing systems, a frame may be further processed to derive a wavefront refractive error, which may characterize the degree of spherical error (e.g., myopia or hyperopia), cylindrical error (e.g., astigmatism), and high order aberration error of the measured eye 100. The processing required, however, is quite complex and may take much longer than the measuring period (e.g., inverse of the frame rate) of the aberrometer 210. Accordingly, the embodiments herein are directed to methods and systems for selecting only a subset of the frames for processing to obtain a corresponding wavefront refractive error.

For example, the illustrated system 300 may be used to train a machine learning model 302 to estimate the quality of a frame (“the subject frame”) based on one or both of (a) a set of contiguous frames including the subject frame and (b) the outputs of one or more imaging modalities for a window including the time of capture of the subject frame (“the subject time”). In particular, the noise present in (a), (b), or data derived therefrom may be processed by the machine learning model 302 to estimate the quality of wavefront refractive error obtained by processing the subject frame.

The machine learning model 302 may be trained using training data 304. The training data 304 may include a plurality of training data entries each including an input and a desired output corresponding to the input. The training data entries may be a set of input time series data and output time series data. For example, for a given time step, the input time series data may include a plurality of values corresponding to the time step and the output time series data may include an output value corresponding to the time step. Alternatively, a training data entry may include a set of time series data including data for a plurality of variables for a plurality of contiguous time steps in a time window and the output of the training data entry may include a single value.

For example, the inputs may include time series data for a plurality of eye position metrics 306. Eye position metrics 306 may be characterized as metrics that characterize movement of the eye 100 itself, the patient's eyelid, or other temporary artifacts that may interfere with obtaining an accurate measurement from the aberrometer 210.

The eye position metrics 306 may include time series data for some or all of the following variables obtained using an imaging modality other than the aberrometer 210:
Corneal vertex position (furthermost corneal point along the visual axis in x, y, and/or z dimensions; the visual axis is a line that crosses the fixation point, e.g., fixation target 204b, and the center of the corneal sphere)
pupil diameter
Pupil center position (x, y, and or z dimensions)
Gaze angle (angle between the device fixation axis and the patient's visual axis of the eye 100)
Chord μ (lateral offset between the cornea apex (on the visual axis) to the pupillary axis)

The above-listed variables may be obtained by analysis of the output of the camera 204, corneal topography device 206, and/or OCT 208.

The eye position metrics 306 may include data that may be obtained from the subject frame itself, such as:
Image geometry (e.g., symmetry) (used to detect whether eyelid is partially closed or light is otherwise partially blocked by cataracts or other conditions)
Specular reflection detection (measures severity of specular reflection of infrared (IR) light from the cornea and/or crystalline lens (for phakic patients) or IOL (for pseudophakic patients) that reaches a detector of the aberrometer).

Variables obtained from the output of the camera 204, aberrometer 210, or one or more other imaging modalities may be further processed to obtain the eye position metrics 306. For example, the eye position metrics 306 may include noise characterizations of any of these values. For example, the eye position metrics 306 may include a noise metric for each variable, such as standard deviation of values for that variable during a time window.

The output of a training data entry may include a value of wavefront refractive error noise 308. For example, a training data entry may include eye position metrics 306 calculated for time series data captured for the eye 100 during the time window as defined above. Aberrometer frames captured for the eye 100 during the same time window (e.g., a window that is at least 80, 90, 95, or 99 percent overlapping) may be processed to obtain the wavefront refractive error for each frame. The time window may encompass at least 5, 10, 50, 100, 150, or more frames output by the aberrometer. A noise metric may then be calculated as a statistical characterization of the wavefront refractive errors for the frames and used as the output for the training data entry. For example, a standard deviation, variance, or other metric of noise. The noise metric may be a statistical characterization of difference values for the wavefront refractive errors, e.g., the difference values each being a difference between (a) a wavefront refractive error of the wavefront refractive errors for the frames and (b) a reference refractive error, such as a subjective refractive error obtained by an optometrist.

The training data 304 may be processed by a training algorithm 310 in order to train the machine learning model 302. For example, the input of each training data entry may be processed using the machine learning model 302 to obtain a prediction. The training algorithm 310 may compare the prediction to the output of the training data entry and update the machine learning model 302 according to the comparison. In the illustrated embodiment, the prediction may be an estimated wavefront refractive error noise based on the eye position metrics 306 of the input.

The machine learning model 302 may be implemented in various ways. In the illustrated embodiment, the machine learning model 302 may include a regression tree fitting stage 312 followed by a regression module 314. However, other approaches may be used such as a neural network, deep neural network, convolution neural network, multiple linear regression model, random sample consensus regression model, multiple polynomial regression model, support vector regression model, Bayesian neural network, genetic algorithm, or any other type of machine learning model.

The training algorithm 310 may validate the machine learning model 302 using validation data 316. The validation data 316 may include training data entries as defined for the training data 304, such as eye position metrics 306 and corresponding wavefront refractive error noise 308 that may be used to assess accuracy of the machine learning model 302 following training.

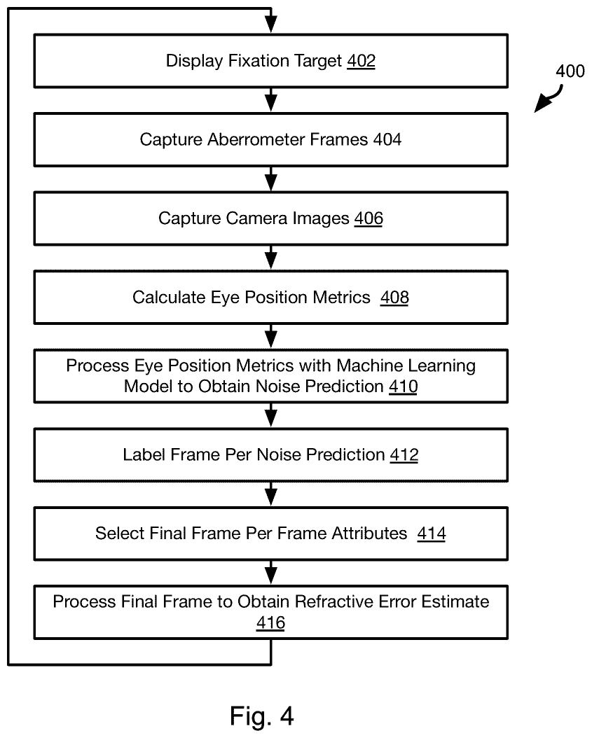

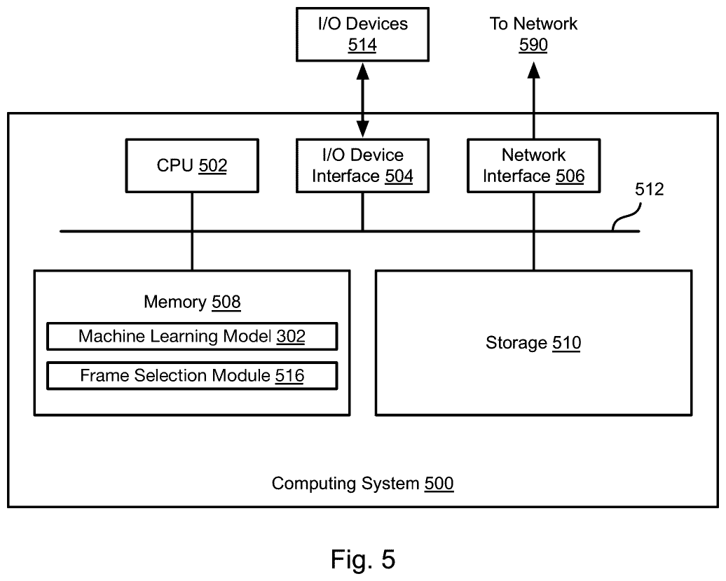

FIG. 4 illustrates an example method 400 that may be performed using the machine learning model 302 in order to select frames from the output of an image modality, such as an aberrometer (e.g., aberrometer 210), and determine a refractive error estimate for an eye of a patient. The method 400 may be executed using a computing system 500 as described below with respect to FIG. 5.

The method 400 may include displaying, at step 402, a fixation target to the eye 100 of a patient, such as using the fixation target 204b of a camera 202 or imaging device according to any imaging modality, such as aberrometer 210.

While displaying the fixation target, the method 400 may include capturing, at step 404, a plurality of aberrometer frames from the aberrometer 210 and capturing, at step 406, images of the eye 100 using a different imaging modality, such as a camera 202. As noted above, steps 404, 406 may include capturing frames and images at a plurality of timesteps. The frame rates of the aberrometer 210 and the camera 202 may be identical or different and may be synchronous or asynchronous.

For subsequent steps of the method 400, “the frames” refers to frames captured at step 404 during a first time window (e.g., between times of capture of first and last captured frames) and “the images” refers to images captured at step 406 during a second time window (e.g., between times of capture of first and last captured images), the second time window at least 80, 90, 95, or 99 percent overlapping the first time window

The method 400 may include calculating, at step 408, eye position metrics for one or both of the frames from step 404 and the images from step 406. The eye position metrics may include some or all of the eye position metrics 306 as defined above. As noted above, the eye position metrics may include a statistical characterization of noise for values of variables measured during the time window.

The method 400 may include processing, at step 410, the eye position with the machine learning model 302 to obtain a noise prediction, e.g., a predicted wavefront refractive error noise as defined above with respect to the wavefront refractive error noise 308.

The method 400 may include labeling, at step 412, the frames from step 404 according to the noise prediction. For example, the first time window may be a sliding time window with a center frame, last frame, or other frame position being the “subject frame” of the frame window, e.g., the frame that will be labeled as a result of processing the frames within the first time window. Accordingly, for each iteration of the method 400, the subject frame may be labeled according to the noise prediction obtained at step 410.

The label may be assigned based on a threshold, e.g., values above a threshold may be labeled as unsuitable. Values below the threshold may labeled as potentially suitable.

The label may be assigned with reference to the noise prediction of adjacent frames, e.g., N contiguous frames before and/or after the frame being labeled. For example, a frame with a noise prediction that is greater than those of adjacent frames by a threshold amount or meeting some other threshold condition may be labeled unsuitable. A frame with a noise prediction that is not greater than those of adjacent frames by the threshold amount or does not meet some other threshold condition may labeled as potentially suitable.

The method 400 may include selecting final frames for which refractive error will be calculated at step 414. Step 414 may include evaluating the labels assigned at step 412 and possibly one or more other values. In particular, attributes of the frames themselves may be considered, such as image geometry (e.g., symmetry) and/or specular reflection as defined above. For example, those with greater image symmetry and/or less specular reflection may be more likely to be selected as a final frame. The selection may be based on density of frames labeled as suitable: where the density is higher, more selectivity may be used to select the final frames based on attributes of the frames and/or frames may be selected at random or at fixed time intervals. Where density is lower, less selectivity may be used.

At step 416, the final frames may be processed to obtain refractive error estimates, such as a wavefront refractive error as defined above. The refractive error estimates may be output to a display device, to a storage device to be stored for later use, or other output modality.

The method 400 may be repeated any number of times. For example, the method 400 may be used to provide real-time feedback during an ophthalmic treatment, such as placement of an intra-ocular lens (IOL).

FIG. 5 illustrates an example computing system 500. The multi-modal imaging device 200 may have some or all of the attributes of the computing system 500.

As shown, computing system 500 includes a central processing unit (CPU) 502, one or more I/O device interfaces 504, which may allow for the connection of various I/O devices 514 (e.g., keyboards, displays, mouse devices, pen input, etc.) to computing system 500, network interface 506 through which computing system 500 is connected to network 590, a memory 508, storage 510, and an interconnect 512.

CPU 502 may retrieve and execute programming instructions stored in the memory 508. Similarly, CPU 502 may retrieve and store application data residing in the memory 508. The interconnect 512 transmits programming instructions and application data, among CPU 502, I/O device interface 504, network interface 506, memory 508, and storage 510. CPU 502 is included to be representative of a single CPU, multiple CPUs, a single CPU having multiple processing cores, and the like.

Memory 508 is representative of a volatile memory, such as a random access memory, and/or a nonvolatile memory, such as nonvolatile random access memory, phase change random access memory, or the like. As shown, memory 508 may store the machine learning model 302 and a frame selection module 516, which may include executable code implementing the method 400 and other functions described herein.

The computing system 500 may include storage 510, which may be non-volatile memory, such as a disk drive, solid state drive, or a collection of storage devices distributed across multiple storage systems.

### Additional Considerations

The preceding description is provided to enable any person skilled in the art to practice the various embodiments described herein. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

A processing system may be implemented with a bus architecture. The bus may include any number of interconnecting buses and bridges depending on the specific application of the processing system and the overall design constraints. The bus may link together various circuits including a processor, machine-readable media, and input/output devices, among others. A user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further. The processor may be implemented with one or more general-purpose and/or special-purpose processors. Examples include microprocessors, microcontrollers, DSP processors, and other circuitry that can execute software. Those skilled in the art will recognize how best to implement the described functionality for the processing system depending on the particular application and the overall design constraints imposed on the overall system.

If implemented in software, the functions may be stored or transmitted over as one or more instructions or code on a computer-readable medium. Software shall be construed broadly to mean instructions, data, or any combination thereof, whether referred to as software, firmware, middleware, microcode, hardware description language, or otherwise. Computer-readable media include both computer storage media and communication media, such as any medium that facilitates transfer of a computer program from one place to another. The processor may be responsible for managing the bus and general processing, including the execution of software modules stored on the computer-readable storage media. A computer-readable storage medium may be coupled to a processor such that the processor can read information from, and write information to, the storage medium. In the alternative, the storage medium may be integral to the processor. By way of example, the computer-readable media may include a transmission line, a carrier wave modulated by data, and/or a computer readable storage medium with instructions stored thereon separate from the wireless node, all of which may be accessed by the processor through the bus interface. Alternatively, or in addition, the computer-readable media, or any portion thereof, may be integrated into the processor, such as the case may be with cache and/or general register files. Examples of machine-readable storage media may include, by way of example, RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The machine-readable media may be embodied in a computer-program product.

A software module may comprise a single instruction, or many instructions, and may be distributed over several different code segments, among different programs, and across multiple storage media. The computer-readable media may comprise a number of software modules. The software modules include instructions that, when executed by an apparatus such as a processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module, it will be understood that such functionality is implemented by the processor when executing instructions from that software module.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. An ophthalmic system for obtaining a refractive error of an eye of a patient, the system comprising:
an aberrometer configured to generate frames including measurements of an eye of a patient; and
a computing device configured to:
receive the frames from the aberrometer;
calculate noise data from at least one of (a) the frames and (b) images of the eye obtained using an imaging modality;
process the noise data to obtain labels for the frames;
select a selected frame from the frames according to the labels;
process the selected frame to obtain the refractive error of the eye; and
output the refractive error.

2. The system of claim 1, further comprising the imaging modality, the computing device further configured to calculate noise data from at least (b).

3. The system of claim 2, wherein the computing device is further configured to calculate noise data from both of (a) and (b).

4. The system of claim 2, wherein the imaging modality is a camera.

5. The system of claim 4, wherein the imaging modality is a three-dimensional camera system configured to capture three-dimensional position coordinates of an object.

6. The system of claim 2, wherein the noise data is derived from one or more metrics describing movement of the eye.

7. The system of claim 6, wherein the one or more metrics include a corneal vertex position.

8. The system of claim 6, wherein the one or more metrics include a pupil center position.

9. The system of claim 6, wherein the one or more metrics include an angle between a fixation axis of the device and a visual axis of the eye.

10. The system of claim 6, wherein the one or more metrics include a lateral distance of an apex of a cornea of the eye to a pupillary axis of the eye.

11. The system of claim 1, wherein the computing device is configured to calculate the noise data from image geometry of the frames.

12. The system of claim 1, wherein the computing device is configured to calculate the noise data from specular reflection present in the frames.

13. The system of claim 1, wherein the computing device is configured to process the noise data using a machine learning model.

14. The system of claim 1, wherein the computing device is configured to select the selected frame based on the labels and one or more attributes of the frames.

15. The system of claim 14, wherein the one or more attributes include image geometry and specular reflection of infrared light.

16. A method for measuring a refractive error of an eye of a patient, the method comprising:
generating, using an aberrometer, frames including measurements of the eye of the patient;
receiving, by a computing device, the frames from the aberrometer;
calculating, by the computing device, noise data from at least one of (a) the frames and (b) images of the eye obtained using an imaging modality;
processing, by the computing device, the noise data to obtain labels for the frames;
selecting, by the computing device, a selected frame from the frames according to the labels;
processing, by the computing device, the selected frame to obtain the refractive error of the eye; and
outputting, by the computing device, the refractive error.

17. The method of claim 16, further comprising capturing the images using the imaging modality and calculating the noise data from at least (b).

18. The method of claim 17, further comprising calculating the noise data from both of (a) and (b).

19. The method of claim 16, wherein the imaging modality is a three-dimensional camera system configured to capture three-dimensional position coordinates of an object.

20. The method of claim 16, further comprising calculating the noise data from one or more metrics describing movement of the eye.
